(19) United States
(12) Patent Application Publication
(10) Pub. No.: US 2022/0170844 A1
(43) Pub. Date: Jun. 2, 2022

(54) METHODS FOR DETERMINING PHOTOSENSITIVE PROPERTIES OF A MATERIAL

(71) Applicant: THE CHEMOURS COMPANY FC, LLC, WILMINGTON, DE (US)

(72) Inventors: CHERYL MARIE STANCIK, KENNETT SQUARE, PA (US); DENISE CONNER, NEWARK, DE (US); TODD ROBERT EATON, WALLINGFORD, PA (US)

(73) Assignee: THE CHEMOURS COMPANY FC, LLC, WILMINGTON, DE (US)

(21) Appl. No.: 17/432,128

(22) PCT Filed: Feb. 19, 2020
(86) PCT No.: PCT/US2020/018803

§ 371 Date: Aug. 19, 2021

Related U.S. Application Data
(60) Provisional application No. 62/809,111, filed on Feb. 22, 2019.

(51) Int. Cl. G01N 17/00; G01N 33/03

(52) U.S. Cl. CPC G01N 17/002; G01N 33/03; G01N 17/004

(57) ABSTRACT

This invention provides methods for determining photosensitive properties of product materials by exposing the product materials to controlled light exposures and measuring for changes in the product materials to quantify light sensitivity.

### FIELD OF THE INVENTION

This invention relates to the field of product and packaging materials, more specifically to methods for determining one or more photosensitive properties of a product material. Further, the invention can be applied to assess the influence of a packaging material on an identified photosensitive product material.

### BACKGROUND

Many product materials contain ingredients such as nutrients, fragrances, colorants, and/or flavors, that can be negatively impacted by exposure to natural, filtered, and/or artificial light, including sunlight, retail light, and light in consumer homes. Examples of product materials include, food, beverages, cosmetics, pharmaceuticals, industrial chemicals, agricultural chemicals, produce, beer, wine, oils, dairy foods. Many different chemical and physical changes can result to these product materials as either a direct or indirect result of exposure to light, which can collectively be defined as photochemical processes. As described in Atkins, photochemical processes can include primary absorption, physical processes (e.g., fluorescence, collision-induced emission, stimulated emission, intersystem crossing, phosphorescence, internal conversion, singlet electronic energy transfer, energy pooling, triplet electronic energy transfer, triplet-triplet absorption), ionization (e.g., Penning ionization, dissociative ionization, collisional ionization, associative ionization), or chemical processes (e.g., disassociation or degradation, addition or insertion, abstraction or fragmentation, isomerization, dissociative excitation) (Atkins, P. W.; Table 26.1 Photochemical Processes. Physical Chemistry, 5^{th} Edition; Freeman: New York, 1994; 908.).

As one example, light can cause excitation of photosensitizer species (e.g., riboflavin in dairy food products) that can then subsequently react with other species present (e.g., oxygen, lipids) to induce changes, including degradation of valuable products (e.g., nutrients in food products) and evolution of species that can adjust the quality of the product (e.g., off-odors in food products).

Understanding the photosensitivity of product materials and packaged product materials is important as they should be protected from changes due to light exposure during their distribution, retailing, and consumer use. Moreover, the photosensitivity of product materials should be understood in order to design packaging materials to protect the photosensitive product materials while contained in the packaging.

Recently developed technology provides robust scientific methods and device to rapidly quantify photoprotective performance of packaging concepts in a way that allows relative comparisons between the packaging concepts relevant to the conditions used for such packaging concepts in their targeted real-world applications. See, for example, commonly owned WO 2013/163421 and WO 2013/162947, the subject matter of which is herein incorporated by reference. The device and methods disclosed in these patent applications allow for the creation of performance design models for packaging materials and concepts, and allow for efficient design of photoprotective packages that achieve the required balance of performance attributes for a given package cost, weight, material usage, or other design requirements. WO 2013/163421 and WO 2013/162947 disclose methods and device for quantification of photoprotective performance of packaging concepts in an accelerated timeframe. In certain embodiments, the device comprises a light source which provides a light beam that impinges upon a photoprotective packaging material before being transmitted to a sample cell comprising a photosensitive entity, such as a photosensitive nutrient. In certain embodiments, the device and methods can be used to generate models for the prediction of photoprotective performance values of untested packaging materials based upon some other known qualitative or quantitative property.

The methods of WO 2013/163421 are useful for consumer packaged goods categories where the photosensitivity of the packaged contents is understood. For example, this method is useful for the protection of dairy products where it is known that riboflavin is the key photosensitive entity present in the dairy products that will cause degration of the product nutrients and sensory quality. Thus the methods presented in WO 2013/163421 allow for the assessment of light protection potential of dairy packages using the knowledge of the key photosensitive species and its behavior as a simple solution under the accelerated light exposure conditions. In WO 2013/163421 a model solution comprising riboflavin is used where its behavior under the light exposure conditions predicts performance of the full product system (e.g., dairy milk).

There are many other light sensitive consumer packaged good products where the materials contained within the product interacting with light are neither known nor isolated from other constituents in the product. Thus the teachings of WO 2013/163421, that are based upon a model or marker to predict performance of a known photosensitive material, cannot be applied.

Thus, in circumstances where the photosensitivity of a material or product material are not known, it is valuable to have a method that allows for the identification and quantification of photosensitive product materials. When there is a product material where it is unknown what species, or multiple species, of the material are interacting with light the methods presented in WO 2013/163421 could not be applied for evaluation for photoprotection performance of a packaging material. More specifically, if the light sensitive species present in a product are unknown, then a marker solution to study and apply the teachings of WO 2013/163421 is also not known.

Examples of such product materials having unknown photosensitivity include, for example, natural products, such as oils, juices, plant milks, wine, beer, spirits, liquors, extracts, or other fermented products, present challenges for evaluation as they are often quite complex in their composition and are not standardized. It may be a challenge to isolate the impacts of light to even a few constituents in a product where there may be a complex interplay of interactions within the constituents. Further, there may be inhomogenity in the natural product that would be difficult to replicate with a simple model system (e.g., multiple phases, chain length distribution in polymers, isomers).

For these reasons, it is desirable to have methods that allow for study of product materials such as consumer packaged goods products to identify their photosensitivity. This includes product materials that have light sensitive entities that are unknown as well as products with multiple entities that may be present in unknown amounts or ratios, such as naturally derived products like oils or juices.

Olive oil is a natural product that can be negatively influenced by exposure to light. As it is a natural product, its composition is subject to variations based upon the region and climate in which the olives were grown, the process by which the oil is harvested from the fruits, and the nature in which the oil is subsequently processed, stored, and packaged. In this example, it can be understood that while there may not be a known marker solution representative of all the variables described above to allow for evaluation using the teachings of WO 2013/163421 for an olive oil, rather using evaluation of the whole olive oil could allow for an innovative approach to determine the photosensitivity of the olive oil products.

Cosmetic products can be formulated from a variety of species, often with some species that are natural products (e.g., plant oils and extracts). These products can have issues in their properties if they receive light exposure, such as changes to the product efficacy or fragrance. While it may not be possible to identify an appropriate marker using the teachings of WO 2013/163421, evaluation of the whole product may be useful to determining photosensitivity of the products.

### SUMMARY OF INVENTION

In one aspect, the present invention provides a method for determining at least one photosensitive property of a product material comprising: (a) providing a product material comprising at least one unknown photosensitive entity; (b) providing a cell to contain the product material at a controlled temperature; (c) providing a stable light source to provide a light beam having a light beam intensity; (d) placing the product material into the cell, rendering a sample cell; (e) placing the sample cell into the light beam; (f) exposing the sample cell to the light beam intensity for at least one duration; (g) measuring the changes to the at least one photosensitive entity contained within the sample cell for at least one duration to generate at least one data point; and (h) utilizing the at least one data point to identify change upon the at least one photosensitive entity.

### BRIEF DESCRIPTION OF THE FIGURES

FIG. 1 is a schematic drawing of an apparatus which can be used according to the invention.

FIG. 2 is a schematic drawing of a cell and sample cell according to one aspect of the invention.

### DETAILED DESCRIPTION

It is to be understood that this invention is not limited to particular embodiments, which can, of course, vary. It is also to be understood that the terminology used herein is for the purpose of describing particular embodiments only, and is not intended to be limiting. Further, all publications referred to herein are incorporated by reference herein for the purpose cited to the same extent as if each was specifically and individually indicated to be incorporated by reference herein.

As used in this specification and the appended claims, terms in the singular and the singular forms “a,” “an,” and “the,” for example, include plural referents unless the content clearly dictates otherwise. Thus, for example, reference to “photosensitive entity,” “the photosensitive entity,” or “a photosensitive entity” also includes a plurality of photosensitive entities. Use of the term “a photosensitive entity” also includes, as a practical matter, many molecules of that photosensitive entity.

Additionally, as used herein, “comprising” is to be interpreted as specifying the presence of the stated features, integers, steps, or components as referred to, but does not preclude the presence or addition of one or more features, integers, steps, or components, or groups thereof. Thus, for example, a sample comprising a photosensitive entity may contain additional photosensitive entities or other components, such as other non-photosensitive nutrients. Additionally, the term “comprising” is intended to include examples encompassed by the terms “consisting essentially of” and “consisting of.” Similarly, the term “consisting essentially of” is intended to include examples encompassed by the term “consisting of.”

The invention provides methods to evaluate product materials which, throughout this disclosure can also be referred to as products, foods, beverages, cosmetics, or other specific classes.

The invention relates to product materials with unknown photosensitivity. While many aspects of the product material may be known, including the basic constitutents, or compositions, of the product material and even the relative amounts of the constituents, the photosensitivity of the product material under the desired conditions is unknown or unquantified.

In one aspect, the present invention provides a method for determining at least one photosensitive property of a product material comprising: (a) providing a product material comprising at least one unknown photosensitive entity and measuring its initial data point; (b) providing a cell to contain the product material at a controlled temperature; (c) providing a stable light source to provide a light beam having a light beam intensity; (d) placing the product material into the cell, rendering a sample cell; (e) placing the sample cell into the light beam; (f) exposing the sample cell to the light beam intensity for at least one duration; (g) measuring the changes to the at least one photosensitive entity contained within the sample cell for at least one duration to generate at least one data point; and (h) utilizing the at least one data point and the initial data point to identify change upon the at least one photosensitive entity.

In an aspect of the invention, the product material comprising at least one unknown photosensitive entity is maintained under one or both of controlled atmosphere conditions and under agitation, the exterior surface of the sample cell is maintained free from condensate, and/or the light beam is collimated. Here controlled atmosphere indicates that the sample cell is closed and, as desired, a gas or atmosphere can be bubbled through the sample to modify the atmosphere within the sample cell. Thus in some embodiments, the headspace of the sample cell or dissolved gases within the sample may contain an atmosphere that is modified and may be different from the ambient atmosphere. This modified atmosphere may contain gases such as nitrogen, carbon dioxide, or oxygen at ratios and levels that are different than that of the ambient atmosphere. In other embodiments additional or different gases may be used to modify the atmosphere.

In some embodiments, the at least one unknown photosensitive entity are constituents of food, beverages, drugs, pharmaceuticals, cosmetics, agricultural chemicals, or other photosensitive-species-containing products. In other embodiments, the at least one unknown photosensitive entity comprise one or more photosensitive entities present in a product material selected from the group consisting of natural and synthetic food additives, dyes, and pigments; chlorophyll; myoglobin, oxymyoglobin, and other hemeproteins; water and fat soluble essential nutrients, minerals, and vitamins; food components containing fatty acids; oils; proteins; pharmaceutical compounds; personal care and cosmetic formulation compounds and components; household chemicals and their components; and agricultural chemicals and their components. In additional embodiments, the at least one unknown photosensitive entity are present in materials selected from 2, 3, 4, 5, 6, 7, 8, 9, 10, or more of the given classes.

The invention can be better understood with reference to the Figures and discussion of certain embodiments according to the invention.

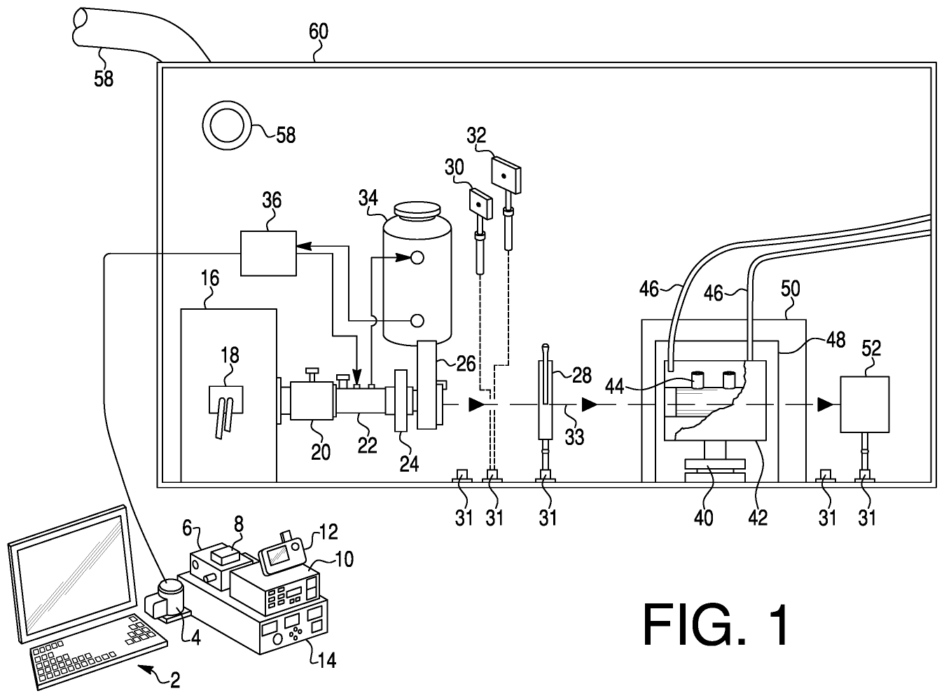

FIG. 1 illustrates one possible embodiment of an apparatus of the present invention which is useful in the disclosed methods. It should be understood that the invention is not limited to the description of certain embodiments which follow. The individual components of the overall apparatus can be contained within an enclosure 60, which is generally light blocking with regard to the spectra being analyzed during an experiment. To maintain proper atmospheric conditions (temperature, humidity, etc.) within the enclosure, the enclosure 60 can possess an exhaust fan and fan trunk 58, which allows the air within enclosure 60 to be cycled at a desired interval and/or rate.

Within enclosure 60, a light source, such as a lamp (not shown) can be contained within lamp housing 16, and connected via appropriate electrical connections (not shown) to a light source power supply 14, which in turn is connected via appropriate electrical connections (not shown) to a lamp controller 10.

The light source is a stable light source. A stable light source, as used herein, is one that provides a consistent spectrum in intensity throughout the wavelengths of the light spectrum. In an aspect of the invention, the stability of the light spectrum is monitored and the intensity tuned as needed to correct for intensity changes with, for example, lamp age. The light source can be any suitable light source to produce the desired light intensity, stability, and spectral characteristics. Depending upon the needs of the experiment, light sources employed may include incandescent light sources, fluorescent light sources, arc discharge lamps, LEDs (light emitting diodes), and/or laser light sources. For example, these light sources include but are not limited to carbon arc, mercury vapor, xenon arc, tungsten filament, or halogen bulbs. In one particular embodiment, the light source is a xenon arc lamp.

In certain embodiments, the light source is capable of providing an intensity of between about 0.001 W/cm² and about 5 W/cm² as measured at the defined monitoring position. In other embodiments, the light source is capable of providing an intensity of at least about 0.001 W/cm², 0.005 W/cm², 0.007 W/cm², 0.01 W/cm², 0.05 W/cm², 0.1 W/cm², 1 W/cm², 2.5 W/cm², or 5 W/cm² as measured at the defined monitoring position. In further embodiments, the light source is capable of providing an intensity of not more than about 0.001 W/cm², 0.005 W/cm², 0.007 W/cm², 0.01 W/cm², 0.05 W/cm², 0.1 W/cm², 1 W/cm², 2.5 W/cm², or 5 W/cm² as measured at the defined monitoring position. In further embodiments, the light source is capable of providing an intensity between about 0.005 W/cm² and about 4 W/cm², between about 0.007 W/cm² and about 3 W/cm², between about 0.01 W/cm² and about 2.5 W/cm², between about 0.05 W/cm² and about 2 W/cm², or between about 0.1 W/cm² and about 1 W/cm² as measured at the defined monitoring position.

In other embodiments, the light source is capable of producing light with a spectral signature of about 200 nm to about 2000 nm. In other embodiments, the light source is capable of providing light at a wavelength of at least about 200 nm, 220 nm, 240 nm, 260 nm, 280 nm, 290 nm, 300 nm, 350 nm, 400 nm, 450 nm, 500 nm, 550 nm, 600 nm, 650 nm, 700 nm, 750 nm, 800, nm, 900 nm, 1000 nm, 1250 nm, 1500 nm, 1750 nm, or 2000 nm. In further embodiments, the light source is capable of providing light at a wavelength of not more than about 200 nm, 220 nm, 240 nm, 260 nm, 280 nm, 290 nm, 300 nm, 350 nm, 400 nm, 450 nm, 500 nm, 550 nm, 600 nm, 650 nm, 700 nm, 750 nm, 800, nm, 900 nm, 1000 nm, 1250 nm, 1500 nm, 1750 nm, or 2000 nm. In still further embodiments, the light source is capable of providing a spectral signature of about 220 nm to about 1750 nm, about 240 to about 1500 nm, about 260 to about 1250 nm, about 290 to about 1000 nm, about 200 to about 400 nm, about 350 to about 750 nm, or above about 750 nm.

In certain embodiments, the intensity and/or spectral characteristics of the light source are controlled and/or modified by one or more of a lens, a water-based infrared filter (to manage the heat signature of the light beam), and a spectral filter. For example, the light from a lamp within lamp housing 16 travels through a collimating lens assembly 20, then through an infrared filter 22, which is a water-based infrared filter attached to water reservoir 34 and water pump 36, the flow volume of which is controlled by a pump flow controller 4 to which water pump 36 is attached via appropriate electrical connections. The collimated and infrared-filtered light then travels through an optical filter holder 24, which can optionally contain an optical filter or filters to attenuate the light beam or portions thereof. Though the lens, infrared filter, and spectral filter(s) are shown in a particular order in FIG. 1, this is not to be taken as an indication that all of these components are required, nor that the indicated order is required. These components could be used in any desired order and/or in any desired combination, including employing none of them in the apparatus and method of the invention.

In certain embodiments, within enclosure 60, a light source, such as a lamp (not shown) contained within lamp housing 16, is connected via appropriate electrical connections (not shown) to a light source power supply 14, which in turn is connected via appropriate electrical connections (not shown) to a lamp controller 10. Lamp feedback monitor 18 is electrically connected to the lamp controller 10. The lamp feedback monitor 18 communicates with the lamp controller 10 which in turn communicates with the light source power supply 14 to adjust the amount of power provided to the light source and/or in order to adjust the intensity of the light emanating from the light source.

In one embodiment, to ensure that the light beam possesses the proper intensity, a light power density sensor 30 can be positioned within the light beam, for instance removably positioned, using one of a plurality of holders 31 located along light path 33. In a preferred embodiment, the light power sensor 30 can be removably positioned within the light beam using a holder and a suitably designed support apparatus. The light power density sensor 30 is attached via appropriate connections (not shown) to the optical energy meter 12. Light power density sensor 30 can be inserted into an appropriate holder, so that a discrete intensity reading can be taken, for instance, prior to the initiation of an experiment and again after the termination of an experiment and/or at times during an experiment. This would allow the intensity of the light beam to be tested both before and after an experiment so that the user can ensure that the power intensity was correctly set and did not significantly increase or decrease throughout the experiment.

In other embodiments, in order to ensure that the light beam possesses the proper spectral characteristics, a spectrometer sensor 32 can be removably positioned within the light beam using one of a plurality of holders 31 located along light path 33 or by using a holder and a suitably designed support apparatus. The spectrometer sensor 32 is attached via appropriate connections (not shown) to a spectrometer 8. Spectrometer sensor 32 can be inserted into an appropriate holder, so that a discrete spectrometry reading can be taken, for instance, prior to the initiation of an experiment and again after the termination of an experiment. This would allow the spectral characteristics of the light beam to be tested before and after an experiment so that the user can ensure that the spectral characteristics were as desired and stable in the time frame of the experiment.

In another embodiment, part of the light beam can be directed away from light path 33 towards a suitable monitoring position (not shown) so as to allow monitoring of the light beam intensity and/or spectral characteristics during an experiment.

Light exposure initiation and cessation during operation of the apparatus or method can be controlled, for example, by a shutter mechanism 26, the operation of which is controlled by a shutter controller 6, to which it is attached via appropriate connections (not shown). Further, the cross sectional area of the light beam impinging upon the sample cell can be adjusted by an iris 28 located within one of the plurality of holders 31, which can be opened and closed as needed to produce a light beam of the desired diameter. Again, though these components are illustrated in FIG. 1, this should not be taken as an indication that one or all of them is required. For instance, the apparatus could be operated without a shutter by simply controlling initiation of the light beam through the lamp controller 10 and/or light source power supply 14. Similarly, the size of the light beam could be alternatively controlled, for example, through the collimating lens 20.

After passing through the iris 28, the light beam will impinge upon the sample cell 44. In an aspect of the invention sample cell 44 can be held in place during the experimental run by sample cell holder 42, which optionally can be insulated so that it retains temperature more efficiently and effectively. Sample cell holder 42 is in direct contact with heat transfer block 48, which is attached to thermoelectric device 50, under the control of thermoelectric controller (not shown). Thermoelectric device 50 can be either a heater or cooler, or a device that is capable of both heating and cooling. During operation, thermoelectric controller directs a temperature set point for thermoelectric device 50. Through heat transfer block 48, the temperature gradient (cold or heat) generated by thermoelectric device 50 is transferred to sample cell holder 42. This allows the temperature within sample cell 44 to be maintained at a fixed temperature throughout an experimental run. Optionally, a heat transfer compound can be used to facilitate heat transfer between the sample cell 44 and the sample cell holder 42. In certain embodiments, the temperature can be set at a temperature between about −20° C. and about 100° C. In other embodiments, the temperature can be set at a temperature of at least about −20° C., −10° C., −5° C., −2° C., 0° C., 1° C., 2° C., 3° C., 4° C., 5° C., 6° C., 7° C., 8° C., 10° C., 25° C., 50° C., or 100° C. In further embodiments, the temperature can be set at a temperature of not more than about −20° C., −10° C., −5° C., −2° C., 0° C., 1° C., 2° C., 3° C., 4° C., 5° C., 6° C., 7° C., 8° C., 10° C., 25° C., 50° C., or 100° C. In still further embodiments, the temperature can be set at between about −10° C. and about 50° C., about −5° C. and about 25° C., about −2° C. and about 10° C., about 0° C. and about 8° C., about 1° C. and about 7° C., about 2° C. and about 6° C., about 3° C. and about 5° C. In certain other embodiments, the temperature is set at about 4° C. In an embodiment, the deviation about the temperature set point is less than 1° C.

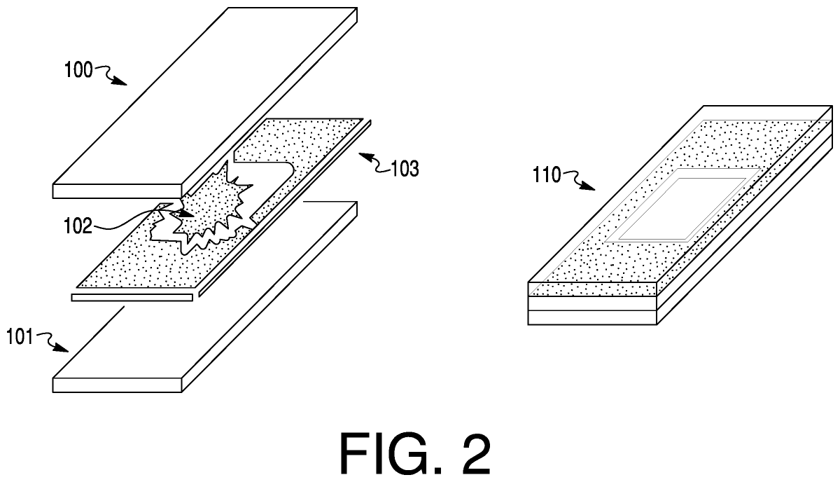

Sample cell for liquid samples 44 can comprise any suitable material and shape such that it possesses the desired optical characteristics. Preferably, sample cell 44 is optically transparent in the spectral range being investigated during the experiment. In certain embodiments, sample cell 44 is made of quartz. In further embodiments, sample cell 44 is made of glass. In certain embodiments sample cell 44 can be substantially flat on one end, thereby allowing the light to impinge upon the sample cell at an angle that is substantially perpendicular to the flat end of the sample cell 44, which can be a desirable optical situation. In an aspect of the invention, the sample cell can be a glass or quartz bottle, jar, or similar shape. In certain embodiments, the sample can be in liquid form, emulsion, or suspended form. In certain embodiments, the sample can be in solid or gel form, such as a cream, paste, powder, or the like. One suitable sample cell for such solid samples is shown in FIG. 2. As shown, the sample cell 110 includes glass plates 100 and 101, which can be positioned to sandwich gasket material 103. Gasket material 103 is provided with a cavity of suitable size where sample material 102 is provided.

In certain embodiments sample cell 44 can also be equipped with one or more access ports (not shown) to allow test samples, additives, or gases to be added or withdrawn from the cell and/or to allow a sample cell thermocouple or other probes or sensors to be inserted into sample cell 44 during an experimental run.

Use of a thermocouple allows the temperature of the cell contents to be monitored and/or controlled throughout an experimental run. In certain embodiments, the thermocouple and/or temperature meter is placed in communication with a thermoelectric controller such that the temperature can be automatically adjusted throughout an experimental run to maintain the material at the desired temperature.

Further, access ports could allow for an optional gas delivery tube and/or atmospheric sensor (not shown) to be inserted into the sample cell during an experimental run for monitoring and/or controlling the atmospheric conditions within sample cell 44 throughout the experimental run. Additionally, directly below insulated sample cell holder 42 is a magnetic stirring motor 40, which is attached via appropriate connections to a magnetic stirrer speed controller (not shown). This allows a magnetic stir bar (not shown) to be located within sample cell 44 during an experimental run so that the magnetic stirring motor can effectuate agitation of the material at a desired speed throughout an experimental run, thereby ensuring substantial material homogeneity.

In certain embodiments, dry air, meaning air with relatively low humidity, can be supplied to the front and/or rear faces of sample cell 44 via delivery tubes 46 in order to prevent or reduce condensation forming on the sample cell. As used herein, the term “air” means atmospheric air or any other suitable gas, such as gaseous nitrogen.

Any light that passes completely through sample cell 44 will eventually impinge upon beam stop 52, which is constructed in such a way that it captures substantially all remaining light without allowing any significant portion of the light to reflect back toward the sample cell.

In certain embodiments, one or more of the components of the overall apparatus may be controlled or monitored by computer 2. This can include one or more of light source power supply 14, lamp controller 10, pump flow controller 4, water pump 36, lamp output feedback detector 18, optical energy meter 12, shutter mechanism 26, shutter controller 6, iris 28, spectrometer 8, a thermocouple (not shown), temperature meter (not shown), thermoelectric controller (not shown), magnetic stirrer speed controller (not shown), gas supply and metering device (not shown) and atmospheric sensor (not shown), air supply (not shown), or pressure regulator (not shown).

During operation of the apparatus disclosed herein, a material comprising at least one photosensitive entities is placed in sample cell 44.

In certain embodiments, the product material of study comprises a photosensitive nutrient or entity. While the details of the content of a product material may be unknown, in particular embodiments, it may be known that the photosensitive entity is selected from:
i. natural and synthetic food additives, dyes, and pigments (e.g., curcumin, erythrosine);
ii. chlorophyll (all variants);
iii. myoglobin, oxymyoglobin, and other hemeproteins;
iv. water and fat soluble essential nutrients, minerals, and vitamins (e.g., riboflavin, vitamin A, vitamin D);
v. food components containing fatty acids, particularly polyunsaturated fatty acids;
vi. oils (e.g., olive oil, soybean oil, etc.);
vii. proteins (e.g., proteins derived from the amino acids tryptophan, histidine, tyrosine, methionine, cysteine, etc.);
viii. pharmaceutical compounds;
ix. personal care and cosmetic formulation compounds and their components;
x. household chemicals and their components; and
xi. agricultural chemicals and their components.

The product material of interest could be studied in neat form or as a component of a solution or formulation. For example, a product material could be diluted or dispersed with a solvating material to facilitate its study. Further a product material could be physically manipulated from its form for evaluation. For example, a pressed cosmetic powder could be milled into a loose powder and placed in a suitable sample cell. In certain embodiments, multiple unknown photosensitive entities could be present in the product material of study, each at different concentrations. Different modes of light-induced change or degradation could occur in the system based upon the chemical nature of the photosensitive entities present to participate in the changes.

For example, for complete food systems, a combination of fats, oxygen, and photosensitive nutrients could be present to allow the interplay between multiple photosensitive entities and associated species to be observed upon light exposure. Thus the product material may be monitored for change with light exposure by one or more methods to track for these impacts. The product material may be monitored by 1, 2, 3, 4, 5, 6, 7, 8, 9 or 10 or more different methods after duractions of light exposure.

The sample cell and product material contained therein are brought to an appropriate temperature for the test, for example a temperature between about −20° C. and about 100° C. Light produced by the light source, which has been optionally collimated, filtered, focused, and/or sized, at a desired intensity (e.g., 0.01-5 W/cm² as measured at the defined monitoring position) and wavelength (e.g., 290-1000 nm) is then made to impinge upon the sample cell and product material contained therein. The temperature of the product material may be studied at ambient or room temperature and the temperature can be monitored to ensure it is held at a constant value.

Because the one or more entities within sample cell 44 are photosensitive, the light impinging upon them will cause some level of change which can be quantified at desired intervals either by measuring the product material while it is contained within sample cell 44 or by removing a test aliquot for measurement by external methods. Suitable analytical methods for determining the amount of light-induced change or degradation include HPLC (high performance liquid chromatography), GC (gas chromatography), IR (infrared) spectroscopy, NMR (nuclear magnetic resonance) spectroscopy, UV-VIS (ultra-violet, visible) spectroscopy, colorimetry, MS (mass spectrometry) coupled with other techniques (e.g., GC-MS and LC-MS), fluorescence spectroscopy, ion chromatography, thin layer chromatography (TLC), analytical wet chemistry, viscosity, dissolved oxygen monitoring, evolution of gasses, chromotagraphy and/or electrochemical analysis (e.g., polarography, voltammetry). In particular embodiments, the measurement method is HPLC based which involves removal of a test aliquot from sample cell 44. In another embodiment, the measurement method is UV-VIS spectroscopy based when product material analysis is performed while it is contained within sample cell 44. In another embodiment the color change to a product material is monitored through the light exposure by removing the sample cell from the light exposure unit and measuring the product material color in a separate instrument.

The product material is monitored before the light exposure begins. Then the product material is subsequently monitored or evaluated after desired length of light exposure, with measurements performed at the desired product material evaluation intervals. The light exposure time and the product material evaluation interval(s) are a function of the product material under investigation, environmental conditions (e.g., temperature and gas modification), and the analytical study of its associated rate of change. In certain embodiments, the total light exposure time is less than 12 hours, less than 11 hours, less than 10 hours, less than 9 hours, less than 8 hours, less than 7 hours, less than 6 hours, less than 5 hours, less than 4 hours, less than 3 hours, less than 2 hours, less than 1 hour, less than 45 minutes, or less than 30 minutes.

The product material evaluation intervals should be selected to obtain a minimum of two data points over the total light exposure, or product material observation duration. It is typically desireable to have a product material evaluation prior to the light exposure and a product material evaluation at the conclusion of the light exposure. Additional product material evaluations can be performed at intervals throughout the duration of the light exposure. Product material evaluations can be performed by removing product material aliquots from the sample cell or by evaluating the product material in situ during the sample light exposure period. The light exposure may be intermittently stopped to allow for a product material to be removed or evaluated in the cell and then the light exposure can be resumed. In particular embodiments, the sampling intervals are selected to obtain at least 2, 3, 4, 5, 6, 7, 8, 9, 10, 11, 12, 13, 14, 15, 16, 17, 18, 19, 20, 25, 30, 35, 40, 45, or 50 data points. In certain embodiments the data points are distributed based on the anticipated product material change rates. Selected intervals will thus be dependent upon the rate of change of the product material, or its photosensitivity. In certain embodiments, the samples are extracted automatically via syringe pump or other suitable device and are delivered directly to vials or analytical equipment for analysis. In certain embodiments, the product material is evaluated in situ in the light exposure apparatus.

Once two or more evaluations have been performed for a light exposure experiment, the resulting data points tracking the change potential of the product material or derivative product(s) can be used to assign a photosenstivity value to the test packaging material. Such photosenstivity values can include, for instance, a pseudo-first order rate constant for light-induced change or degradation of the photosensitive entity being examined which can be converted to a Light Sensitivity Factor (LSF) via a suitable mathematical transformation. For example, LSF could be defined as the half-life of a photosensitive entity which is calculated, for example, for pseudo first order reaction kinetics by dividing In(2) by the obtained pseudo-first order rate constant. Moreover, by regulating the variables of the experimental runs, such as light spectra, light intensity, light focus, duration of light exposure, sample temperature, sample homogeneity, and sample atmospheric conditions, results can be obtained with sufficient accuracy and precision to allow for quality run-to-run comparisons to be made.

### EXAMPLES

### Example 1

Two skincare cosmetic products were purchased at retail in the United States:
a) L'Occitane Eau d'Immortelle toner product (purchased at L'Occitane store); and
b) Neogen Green Tea foaming face wash (purchased at Sephora).

These two products were chosen based on their potential for light interaction based on the package appearances, claims, and ingredients. Both products are translucent fluids and could be easily transferred into vials to study the effect of light exposures.

These products are both packaged in translucent plastic bottles. Neither of these two packages had a secondary carton (e.g., an outer paperboard box) and thus the bottle and closure were the only packaging components providing for light protection in the retail environment.

### Laboratory Evaluation of Cosmetic Products

The products were first assessed to determine if spectral signatures could be monitored. For this evaluation, aliquots of each cosmetic product were transferred into quartz cuvettes for UV-Vis spectral analysis. For the UV-Vis analysis, a HunterLab UltraScan PRO spectrophotometer (instrument serial #USP1274) was used in total transmittance (TTRAN) mode setup for liquid cell measurements with 1 cm path length (5×5 cm cell face area) (instrument serial #USP1274). A cuvette filled with deionized water was used as a reference sample for these measurement. As needed, when spectral features were saturated in their response samples were diluted with deionized water. Thus, the Neogen product was diluted 5:1 by weight (water:Neogen) in deionized water while the L'Occtaine product was studied without dilution.

Approximately 40 mL of each sample was transferred into a square glass jar (60 mL total volume with 3 cm square footprint). The square glass jar was placed into a light exposure apparatus, depicted in FIG. 1 and described in commonly owned WO2013/162947, on a platform at a position (31, near 28) the instrument light beam (33) for an intense light exposure. During the light exposure, the sample was vigorously stirred with a magnetic stir bar. The width of the bottle was fully encompassed within the light beam (33). Based on calculations of the light power density and attenuation at defined instrument locations, an illuminance measurement of about 290,000 lux is applied at the position in the light beam where the glass jar was placed for exposure. The sample temperature was unregulated but held at approximately constant ambient lab temperature (19° C.) during the light exposure duration.

After durations of light exposure, portions of the product sample were removed from the bottle for off line UV-Vis spectral analysis.

The spectra for the L'Occitane Eau d'Immortelle toner product showed minor to no change after the 120 minute intense light exposure. Thus for this product there are no significant light sensitive features to be readily monitored using this detection approach in the normal measurement time scales. No further research was conducted on this product.

The diluted Neogen product spectra showed light sensitivity in features at 370, 390, 400, 480, and 670 nm.

### Reaction Kinetics Modeling

In applicants' standard method for the measurement of light protection performance as disclosed in WO 2013/163421, a riboflavin marker solution can be monitored for decay after durations of light exposure. The concentration of riboflavin as a function of light exposure time is tracked and its loss has been found to follow first order reaction kinetics; more precisely, pseudo-first order decay of riboflavin. If light intensity were changed, it would also have an effect on the reaction kinetics, but because intensity is held constant first order behavior is effectively observed, or pseudo-first order decay. Here this approach to apply a rate law to characterize changes to a light exposed product is applied.

The integrated first order rate law where [A] indicates the concentration of reactant A at time t is as follows:

$${ln [ A ]}_{t} = - k t + {ln [ A ]}_{0}$$

In this rate law the pseudo-first order rate constant, or simple rate constant, is k and [A]₀ is the initial concentration of A. To assess for first order reaction kinetics, data is plotted with In[A] as y and t as x. When first order reaction kinetics describe the concentration profile of the species A, a straight line will be observed with a slope of −k.

Further, for first order reaction kinetics the half-life, or time when [A]ₜ=[A]₀/2, is defined as:

$${t}_{1 / 2} = \frac{ln ( 2 )}{k} \qquad$$

This approach was applied to the diluted Neogen product. Analysis of the spectral features of the diluted Neogen product showed they decayed with reasonable approximation to first order reaction kinetics (Table 1.1). While this model provides a reasonable approximation of the observed data, there is some discrepancy with this reaction kinetics model.

TABLE 1.1
Neogen Face Wash First Order Reaction Kinetics
Decay Model

| | wavelength | | | | |
|---|---|---|---|---|---|
| | 370 nm | 390 nm | 400 nm | 480 nm | 670 nm |
| k (−m) (1/min) | 0.006 | 0.008 | 0.008 | 0.008 | 0.010 |
| b | −0.09 | −0.11 | −0.12 | −0.05 | −0.19 |
| Ao (exp(b)) | 0.91 | 0.89 | 0.89 | 0.95 | 0.83 |
| t½ (min) | 108.7 | 91.0 | 85.4 | 90.9 | 70.0 |
| R\hat{ }2 | 0.953 | 0.949 | 0.951 | 0.979 | 0.922 |

Based on this observation, a second order reaction kinetics model was considered. Using the sample variables described earlier, the integrated second order rate law is as follows:

$$\frac{1}{{[ A ]}_{t}} = k t + \frac{1}{{[ A ]}_{0}}$$

To assess for second order reaction kinetics, data is plotted with 1/[A] as y and t as x. When second order reaction kinetics describe the concentration profile of the species A, a straight line will be observed with a slope of k. Further, for second order reaction kinetics the half-life (or time when [A]ₜ=[A]₀/2) is defined as:

$${t}_{1 / 2} = \frac{1}{{k [ A ]}_{0}}$$

The second order model was applied to the data and excellent correlation coefficients were observed (Table 1.2) for each of the spectral features analyzed; all R² values were above 0.99 indicating the agreement of the data to this model.

TABLE 1.2
Neogen Face Wash Second Order Reaction
Kinetics Decay Model

| | wavelength | | | | |
|---|---|---|---|---|---|
| | 370 nm | 390 nm | 400 nm | 480 nm | 670 nm |
| k (m) (1/min) | 0.010 | 0.014 | 0.017 | 0.056 | 0.164 |
| 1/Ao (b) | 1.004 | 1.063 | 1.083 | 3.892 | 7.776 |
| t½ (b/m) (min) | 98.192 | 73.729 | 65.460 | 69.374 | 47.333 |
| R\hat{ }2 | 0.994 | 0.996 | 0.998 | 0.997 | 0.993 |

Based on the success of monitoring changes to spectral features during the exposure time and the ability to model the observed decay behavior with second order reaction kinetics, we selected this diluted Neogen product as a good candidate for further evaluation using the sample cell (44) in the instrument for insitu monitoring during light exposure described in WO2013/163421.

### In-Situ Monitoring of Light-Exposed Skincare Products

Using the light exposure instrument described in WO2013/162947, the Neogen product was placed in the normal sample cell (44) at a 2:1 dilution ratio, diluted with deionized water by weight. A light intensity of 0.48 W/cm² was used for a 120 minute light exposure at a 19° C.

Using these conditions, a series of experiments was conducted. First an experiment was conducted to evaluate the photosensitivity of the product material under the test conditions denoted “No Package” in Table 1.3.

With the photosensitivity confirmed using the “No Package” condition, further experiments were performed with different packaging materials to assess their light protection performance. A fresh diluted Neogen product sample was used for each experiment. For the packaging materials, we explored three additional conditions: the Neogen commercial package, PET experimental Bottle B, and PET experimental Bottle C each placed into the light beam at the package sample position 28 using the package sample holder described in the examples and drawings of WO2013/162947. We monitored the 670 nm spectral feature for the analysis identified as described. As the relative rate of change of the peak in the data of interest rather than the actual magnitude of the absorbance data, the absorbance data is normalized using the initial time data.

In the analysis, the impact that the package has on the decay of the 670 nm spectral feature is evident. As the package becomes more protective, the changes to the 670 peak are modulated. For the most protective packaging (Bottle C), no measureable change is observed for the 120 minute light exposure. In all other packages, including the commercial package, significant decay is seen in the product spectral features. Based on the exploratory work, the second order reaction kinentics model was applied to determine parameters as reported in Table 1.3.

In addition to the parameters of the second order decay model observed, we report a metric called the Light Protection Factor (LPF), as the half-life calculated with the second order model (t ½=1/(k*A₀)=b/m). Complete data from the series of packages analyzed using the second order model is shown in Table 3.

TABLE 1.3
Parameters for the Light Protection Packge
Performance Evaluation of Neogen Product using a Second Order
Reaction Kinetics Model for the 670 nm Spectral Feauture

| | No | Commercial | | |
|---|---|---|---|---|
| Package | Package | Package | Package B | Package C |
| k (m) (1/min) | 4.44E−02 | 1.38E−02 | 5.11E−03 | 5.31E−05 |
| LPF or t_{1/2} (b/m) (min) | 1.10E+02 | 4.36E+02 | 1.12E+03 | 1.21E+05 |
| R² | 0.997 | 0.989 | 0.988 | 0.012 |

The data where significant degradation occurs in the product yield excellent correlations coefficients all above 0.98; however, when virtually no degradation occurs for package C, the correlation coefficient is poor. This indicates a very high light protection performance for this package that cannot be quantified under the experimental conditions used.

The light protection performance can be considered by looking at the LPF values for this product and package combination. The LPF of the commercial package is LPF 436 min. It is increased an order of magnitude to LPF 1,120 min for Package B and three orders of magnitude to LPF 121,000 min for Package C. Thus the Package B and C designs enhance the light protection performance of the package dramatically as comparted to the commercial package.

This example shows that evaluations of cosmetic products, or other unknown samples, can be performed using applicants' light protection performance assessment instrument with a modified approach to that presented in WO 2013/163421. In summary, in this approach shown in the example, key features in a product material with an unknown photosensitivity were identified. These features were then tracked through the light exposure, either in situ or ex situ, and modeled using an appropriate reaction kinetics model.

For the Neogen product, we identified the photosensitivity of the unknown product. Specifically, we found the product features decayed with second order reaction kinetics with excellent correlation coefficient. We then used the knowledge of the photosensitivity of the product to perform light exposure evaluations with packaging materials to quantify how packaging could provide light protection to the product. Finally, we further use this information to define how a package could be designed to provide additional light protective performance to the product. Such light protection would prevent or limit product changes to products within such packages after receiving light exposures.

### Example 2

Olive oil (Bertolli) was purchased at retail and stored in a dark cabinet prior to the experiment. The olive oil was evaluated using the instrument described in WO2013/162947 by placing the olive oil into the sample cell 44. Temperature was controlled at 20° C. and the olive oil was stirred rigorously throughout the experiment. Packaging samples were cut out of the commercially available olive oil bottles, both PET and glass packaging materials. The packaging sample being tested for light protection was affixed to the sample holder 28 and placed in the sample test position 31 between the test cell and the exposure lamp.

UV/Vis absorbance spectra of the olive oil were collected once a minute for 40 to 90 minutes. Photosensitivity of the product was identified in a peak at 670 nm and was tracked to determine the rate of decay. The degradation of the species at 670 nm followed pseudo-first order kinetics and the data fit to a pseudo-first order reaction model with a rate constant k′ and t_{1/2}, as described in Example 1.

The spectra showed that as the exposure time was increased the absorbance at 670 nm decreased. The absorbance at 670 nm over time was chosen as the spectral feature for pseudo-first order parameter fitting because the absorbance was within the range appropriate for correlating absorbance and concentration (a.u.<1.0), as determined by the Beer-Lambert Law.

The origin of the 670 nm peak was attributed to chlorophyll and a calibration using authentic standards of chlorophyll a in oleic acid was used to determine the chlorophyll concentrations in the olive oils (Table 2.1) showing that as light exposure time increases chlorophyll concentration decreases.

TABLE 2.1
Chlorophyll concentrations as a function of exposure
time,

| | Chlorophyll | Chlorophyll (% of |
|---|---|---|
| Time (minutes) | concentration (mg/kg) | initial) |
| 0 | 16.8 | 100% |
| 5 | 16.6 | 99% |
| 15 | 15.6 | 93% |
| 30 | 14.7 | 87% |
| 45 | 13.8 | 82% |
| 60 | 13.0 | 77% |
| 120 | 11.7 | 70% |
| 150 | 10.6 | 63% |
| 180 | 9.5 | 57% |

The data of Table 2.1 was fit to a pseudo-first order rate decay with a strong correlation observed (R² of 0.993).

With the photosensitivity of the olive oil identified, additional evaluation of commercial packaging samples were conducted in this manner to determine the t_{1/2} during olive oil light exposure, those results are shared below in Table 2.2.

TABLE 2.2
Pseduo-first order rate constants and t_{1/2} values for
tested packaging samples

| Package | Material | Color | k′ (min⁻¹) | t_{1/2} (h) |
|---|---|---|---|---|
| A | PET | Green | 0.00144 | 8.04 |
| B | PET | Dark Green | 0.00075 | 15.60 |
| C | Glass | Dark Green | 0.00171 | 6.77 |
| D | PET | Light Green | 0.00297 | 3.89 |

The data of table 2.2 demonstrate that the methods of the present invention are useful to identify the sensitivity of an unknown product material. Once identified using the method, the product can further be studied to discriminate and quantify the performances of different packaging materials. Here commercial packages for olive oil were studied with this method. This method allows for packages made of different materials and are different colors and quantitatively compared to determine their light protective performance. This method allows for light protection performance to be quantitatively assessed across different packaging formats.

### Example 3

Commercially available cosmetic creams were purchased from retail stores for evaluation to determine their light sensitivity. For evaluation in the light exposure device described in WO2013/163421, as shown in FIG. 2, cosmetic creams 102 were spread onto a glass slide 101 fitted with a neoprene gasket 103, then a second glass slide 100 is placed over the cream 102 and the assembly is taped together to form a sealed chamber of cream with a smooth surface. Two sample assemblies were made, one to be exposed to light (referred to as the “light” sample) and one to be kept in the dark (“dark” sample). Both assemblies are then loaded on the sample holder 28, with the dark sample being positioned behind the light sample and a piece of aluminum foil. This protocol ensures that the samples do not significantly dry during the experiment, and that the dark sample is subjected to the same conditions (i.e. handling, temperature) as the light sample, with the exception of light exposure. The samples were then placed within the test apparatus and exposed to light. The samples were removed periodically for color measurements.

Color measurements were collected on a Hunter Associates Lab Labscan Spectro Colorimeter. Tristimulas XYZ and L*a*b* values were recorded as a function of exposure time, and ΔE* was calculated based on the following equations:

$$Δ {L}^{*} = {L}_{0}^{*} - {L}_{i}^{*} \\ Δ {a}^{*} = {a}_{0}^{*} - {a}_{i}^{*} \\ Δ {b}^{*} = {b}_{0}^{*} - {b}_{i}^{*} \\ Δ {E}^{*} = \sqrt{{( Δ {L}^{*} )}^{2} + {( Δ {a}^{*} )}^{2} + {( Δ {b}^{*} )}^{2}}$$

Where the subscript 0 represent the value at time=0 minutes, and subscript i represents the value at time=i minutes. This measure accounts for all color change, regardless of whether the mode of change is darkening, lightening, yellowing, etc. The difference in ΔE* between “light” and “dark” samples was used to determine the change due to light exposure, with the results shown in Table 3.1 below for light exposure of 60 minutes.

TABLE 3.1
Color change for Creams A-H with no light protection

| | Cream | Cream | Cream | Cream | Cream | Cream | Cream | Cream |
|---|---|---|---|---|---|---|---|---|
| Sample: | A | B | C | D | E | F | G | H |
| ΔE*_{light}- | 19.4 | 2.69 | 11.0 | 7.61 | 6.96 | 2.77 | −0.07 | 0.96 |
| ΔE*_{dark} | | | | | | | | |

In most cases the light-exposed sample changed color more than the dark sample, indicating that light exposure led to the color degradation.

Cream A was studied further by tracking the color change over time with various packaging samples between the cream and the incident light placed at position 28. The color change as a function of time is shown in Table 3.2.

TABLE 3.2
ΔE* of Cream A over time with various package
samples for light protection.

| | Package: | | | | | |
|---|---|---|---|---|---|---|
| | None | E | F | G | H | I |
| Time | ΔE* | ΔE* | ΔE* | ΔE* | ΔE* | ΔE* |
| 0 | 0.0 | 0.0 | 0.0 | 0.0 | 0.0 | 0.0 |
| 5 | 7.6 | 0.5 | 0.4 | 0.5 | 0.1 | n.d. ^{a} |
| 10 | 10.4 | 1.5 | n.d. ^{a} | 0.9 | 0.1 | n.d.^{a} |
| 15 | 13.0 | 3.9 | 3.6 | 1.8 | 0.2 | 0.2 |
| 30 | 16.3 | 7.9 | 6.8 | 6.2 | 0.3 | 0.3 |
| 45 | 18.4 | 9.6 | 8.9 | 7.5 | 0.4 | 0.4 |
| 60 | 19.3 | 11.2 | 10.0 | 8.4 | 1.2 | 0.3 |

^{a} n.d. = not determined.

For cosmetic cream applications, it is desirable to have stable color that is not changing. Generally in the color industry, ΔE values of greater than 1 are perceptible by the human eye. Inspection of the data shows that without any protective packaging (“None”) the cream rapidly changes color during light exposure, eventually plateauing around a ΔE* of 19 after 45 minutes.

Placing light-protective packages between the cream and the incident light decreases the rate of color change. More specifically, Package H limits color change through 45 minutes and package I is able to limit color change through 60 minutes of light exposure.

Thus using the methods of the present invention it was found that product materials could be tracked to assess for photosensitivity. Once identified, further evaluation of package materials could be conducted showing how packages could be discriminated for their light protection performance using such approaches.

1. A method for determining at least one photosensitive property of a product material comprising: (a) providing a product material comprising at least one unknown photosensitive entity; (b) providing a cell to contain the product material at a controlled temperature; (c) providing a stable light source to provide a light beam having a light beam intensity; (d) placing the product material into the cell, rendering a sample cell; (e) placing the sample cell into the light beam; (f) exposing the sample cell to the light beam for at least one duration; (g) measuring the changes to the at least one photosensitive entity contained within the sample cell for at least one duration to generate at least one data point; and (h) utilizing the at least one data point to identify change upon the at least one photosensitive entity.

2. The method of claim 1, wherein the product material is maintained under controlled atmosphere conditions.

3. The method of claim 1, wherein the product material is maintained under the atmosphere of an inert gas conditions.

4. The method of claim 1, wherein the product material is maintained under the atmosphere of nitrogen gas conditions.

5. The method of claim 1, wherein the product material is maintained at a controlled temperature between about −20° C. and 100° C.

6. The method of claim 1, wherein the light source generates a controlled light beam intensity of between about 0.01 and about 5 W/cm².

7. The method of claim 1, wherein the light source generates a light beam with a controlled spectral signature of between about 290 and 1000 nm.

8. The method of claim 1, wherein the product material comprises one or more unknown photosensitive entities selected from one or more of the following classes:
i. natural and synthetic food additives, dyes, and pigments;
ii. chlorophyll;
iii. myoglobin, oxymyoglobin, and other hemeproteins;
iv. water and fat soluble essential nutrients, minerals, and vitamins;
v. food components containing fatty acids;
vi. oils;
vii. proteins;
viii. pharmaceutical compounds;
ix. personal care and cosmetic formulation compounds;
x. household chemicals and their components; and
xi. agricultural chemicals and their components.

9. The method of claim 8, wherein the product material comprises one or more unknown photosensitive entities selected from two or more of the classes.

10. The method of claim 8, wherein the oil comprises olive oil.

11. The method of claim 8, wherein the unknown photosensitive entity comprises a cosmetic formulation compound.

12. The method of claim 1, wherein the unknown photosensitive entity comprises a natural product.

13. The method of claim 1, wherein the cell comprises quartz.

14. The method of claim 1, wherein the cell is in the form of a bottle.

15. The method of claim 1, wherein the cell has at least one substantially flat surface.

16. The method of claim 15, wherein the cell comprises two flat surfaces and the sample is sandwiched between the two flat surfaces.

17. The method of claim 1, wherein the sample is diluted with an appropriate matrix to identify the signature changes.

18. The method of claim 1, wherein the cell is in the form of a jar.
